SEQUENCE LISTING (4 sequences)

SEQ ID NO: 1 - DNA, 1962 bp, Artificial Sequence
  Nucleic Acid
acaaatagcgtccaagagaacattgtaatgaatccttctgataattgtctgatgagcgatgtcatgaaagtcaacctgtctcgtcgtaaaattctgcagtttggcggggcaatgggtgctatcgccctgctgcctgcggcttttggttcgcgcagcgcatttgcgaacccaagccagccattgcagatccgtaaaattacaaatctcacgtttacgtctattccgtttagtaccgaagatcgtgtcatcgttccaaaaggctatagtgcgaaggcgttctatgcctggggcgacccggtcggtctggaaaataataacccgcagtttaaagttgacgcgtcaaactcggcagaagaacaggccgctcaggcgggcatgaaccacgatgggatggcgtactttcctttcgccgaacatggtaacgaacatggcctgctggtgatgaaccatgaatacattgacaacggtctcttgtttcctgatggggataaaacgtggagcctggataaggtgaaaaaatcgcagaacgctatgggcatctcggtgattgaaattaaaaaagtgaaccagcagtgggaagtggtccgcccgagcaaatatgcccgtcgtattacgccgcatacccctatgcgtctgacagggccggcgaaacataacgaactgatgaaaactgtagccgaccctttaggggagtttgtgctggggaccatgcagaactgtgcaaatggtgaaacaccctggcgcacttatctgacctgtgaagagaactggtccgatatttttgtgcgtgaatccggcgacttcacgaagctggataagcgttacgggattatgaagaaagaaaaagaagataagtaccgttggaatgaattcgatgaacgttttaatacggataaacatccgaacgaaccgcaccgcttcggctgggttgttgaaattgacccctttgatccaaacagcaccccggttaaacacaccgccctgggccgcttcaaacacgagggcgccatgctggtgctgagcaaggaagggcacgcagtagtctacatgggtgacgatcagcgtttcgagtacatctataagttcgtttctaaaggtaaatacaacccggcagatcgtgcagcaaacatgtatcttctgtccgaaggcacactctatgtcgcccattttaacgaagataacaccggcgaatggcgtccgctggtccataatcagaacgggctgactgcggagaatggctttctgaatcagggcgatgtgaccatcaaggcacgtatggcggctgatgtggtgggcggcactaaaatggatcgcccggaatggatcgccgtcgatccatatcagactggctctgtgtattgcacccttacaaacaatagccagcgtggtaccgaaggcaaggcgggtattgatgccgccaacccgcgcgttaagaactcgtatggacatatcattcgctggcaagagaatgaccaagattacctgtccgagaccttctcatgggatatcttcgcgctgggcggtaataaatccaagggcgaaaatcatgtgaacggtgatgatttcggttccccggacggactccgtttcgataatcacggcatcttgtgggttcaaacggacgtgtcgagttccacgttaaataaaaaggcctatgaaggcatgggtaataaccaaatgctggcggtaattccagagcagggcgagtttaaacgttttttaacggccccgaacggctcagaagtaaccggtatcgctttcactcctgacaacaaaaccatgtttatcaatattcagcatccgggtgaaccagatagcggcgtgaccgaaccagatcaagtaacagccatttccacctggccggaccgtcaaggtaaaacccgccctcgctctgctaccgtggttattcagaaggaagatggtggcgttatttcatctctcgagcaccaccaccaccaccac

SEQ ID NO: 2 - protein, 1143 aa, Artificial Sequence
  Recombinant Fusion Protein
MMNDGKQQSTFLFHDYETFGTHPALDRPAQFAAIRTDSEFNVIGEPEVFYCKPADDYLPQPGAVLITGITPQEARAKGENEAAFAARIHSLFTVPKTCILGYNNVRFDDEVTRNIFYRNFYDPYAWSWQHDNSRWDLLDVMRACYALRPEGINWPENDDGLPSFRLEHLTKANGIEHSNAHDAMADVYATIAMAKLVKTRQPRLFDYLFTHRNKHKLMALIDVPQMKPLVHVSGMFGAWRGNTSWVAPLAWHPENRNAVIMVDLAGDISPLLELDSDTLRERLYTAKTDLGDNAAVPVKLVHINKCPVLAQANTLRPEDADRLGINRQHCLDNLKILRENPQVREKVVAIFAEAEPFTPSDNVDAQLYNGFFSDADRAAMKIVLETEPRNLPALDITFVDKRIEKLLFNYRARNFPGTLDYAEQQRWLEHRRQVFTPEFLQGYADELQMLVQQYADDKEKVALLKALWQYAEEIVRTGGSGGGSGGGSGTNSVQENIVMNPSDNCLMSDVMKVNLSRRKILQFGGAMGAIALLPAAFGSRSAFANPSQPLQIRKITNLTFTSIPFSTEDRVIVPKGYSAKAFYAWGDPVGLENNNPQFKVDASNSAEEQAAQAGMNHDGMAYFPFAEHGNEHGLLVMNHEYIDNGLLFPDGDKTWSLDKVKKSQNAMGISVIEIKKVNQQWEVVRPSKYARRITPHTPMRLTGPAKHNELMKTVADPLGEFVLGTMQNCANGETPWRTYLTCEENWSDIFVRESGDFTKLDKRYGIMKKEKEDKYRWNEFDERFNTDKHPNEPHRFGWVVEIDPFDPNSTPVKHTALGRFKHEGAMLVLSKEGHAVVYMGDDQRFEYIYKFVSKGKYNPADRAANMYLLSEGTLYVAHFNEDNTGEWRPLVHNQNGLTAENGFLNQGDVTIKARMAADVVGGTKMDRPEWIAVDPYQTGSVYCTLTNNSQRGTEGKAGIDAANPRVKNSYGHIIRWQENDQDYLSETFSWDIFALGGNKSKGENHVNGDDFGSPDGLRFDNHGILWVQTDVSSSTLNKKAYEGMGNNQMLAVIPEQGEFKRFLTAPNGSEVTGIAFTPDNKTMFINIQHPGEPDSGVTEPDQVTAISTWPDRQGKTRPRSATVVIQKEDGGVISSLEHHHHHH

SEQ ID NO: 3 - DNA, 3042 bp, Artificial Sequence
  Exonuclease/Phosphatase
agatctcgatcccgcgaaattaatacgactcactataggggaattgtgagcggataacaattcccctctagaaataattttgtttaactttaagaaggagatatacatatgatgaatgatggtaaacagcaatctacctttctgttccacgactatgaaaccttcggtactcacccggccctggatcgcccggcacagttcgctgcgatccgtaccgactccgaatttaacgtgatcggtgaaccggaggtattctactgcaaaccggcggacgactacctgccacagccgggtgcggtgctgattaccggtatcactccgcaagaagctcgtgccaaaggtgagaatgaagcggcgtttgccgcgcgtatccatagcctgttcaccgtaccgaaaacctgcatcctgggttacaacaacgtgcgtttcgacgacgaagtgacccgtaacatcttctaccgtaacttctatgacccatacgcatggtcctggcagcacgacaacagccgttgggatctgctggatgtaatgcgtgcgtgctatgctctgcgcccagaaggtattaactggccggagaacgacgacggcctgccgagcttccgtctggagcacctgaccaaagcgaacggtatcgaacactccaacgcgcacgatgcgatggcagacgtctatgctactatcgctatggcaaagctggttaaaacccgtcagccgcgcctgtttgactatctgtttacccaccgtaacaaacacaaactgatggctctgatcgacgttccgcagatgaagccgctggttcatgtgtctggtatgtttggtgcttggcgcggcaacacctcttgggtagccccgctggcctggcacccggagaaccgtaacgctgtgatcatggtggacctggcgggtgatatctccccgctgctggaactggactctgacacgctgcgtgaacgtctgtataccgcaaaaaccgatctgggtgataacgccgcagttccggtgaagctggtgcacatcaacaaatgtccggtcctggctcaggcgaataccctgcgtccggaagacgcggaccgtctgggtattaaccgtcagcattgcctggacaacctgaaaattctgcgcgaaaacccgcaggtccgcgaaaaagttgtagccatcttcgcggaagcggaaccgtttaccccatccgacaacgttgacgctcagctgtacaacggcttcttttccgatgcggaccgcgcagcgatgaaaattgttctggaaaccgaaccgcgcaacctgccggcactggatatcactttcgtcgacaaacgtatcgaaaaactgctgttcaactatcgtgctcgtaactttccgggtactctggattacgctgagcaacagcgttggctggaacatcgtcgtcaggtatttaccccggaattcctgcagggctatgcagatgaactgcagatgctggtacaacagtacgcagacgataaggagaaagtggcgctgctgaaagcactgtggcagtacgcggaagaaattgttcgtacgggcggctccggtggcgcgagcggcggttccggcggtcatatggaagaagataaagcatactggaacaaagacgcgcaggatgccctggacaaacagctgggtatcaaactgcgtgaaaaacaggccaaaaacgtgattttcttcctgggtgatggtatgagcctgtccacggttactgcggcgcgtatctataaaggcggtctgactggtaaattcgaacgtgaaaaaatctcttgggaagagttcgacttcgcagccctgtctaaaacttataatacggataaacaggttacggattctgctgcttctgcaaccgcttatctgaccggcgttaagaccaaccagggtgttattggtctggacgctaacaccgttcgtaccaactgctcttaccagctggatgaaagcctgtttacctacagcatcgcacactggttccaggaagctggtcgcagcaccggtgttgtgacctccacccgtgttacccacgctactccggcgggcacctacgcgcacgtagcagatcgcgattgggaaaacgacagcgacgtagtacatgatcgtgaagacccggaaatttgtgacgatatcgcagaacagctggtattccgtgagccgggcaaaaactttaaagtaatcatgggtggcggtcgtcgcggtttcttcccggaagaagcgctggacatcgaagatggtatcccgggtgagcgtgaagacggtaaacacctgatcactgactggctggatgacaaggcttcccagggtgcaactgcatcctacgtatggaaccgtgatgacctgctggcggtggacatccgcaacactgattacctgatgggcctgttcagctacacgcacctggacaccgttctgacccgtgatgccgaaatggacccgactctgcctgagatgactaaagtggccatcgaaatgctgaccaaagacgaaaatggtttctttctgctggtagaaggcggtcgcattgaccacatgcaccacgcgaaccagatccgtcagtctctggctgagaccctggacatggaggaggccgttagcatggcgctgagcatgactgatccggaagaaacgatcatcctggttaccgctgatcacggtcatacgctgactatcaccggttacgcggaccgtaacacggatattctggatttcgctggcatcagcgatctggacgaccgtcgctacactatcctggattacggttctggtccgggttaccacatcactgaggacggcaaacgctacgaaccgactgaagaggatctgaaagatatcaatttccgctacgcgtctgcagcaccaaaacattctgttacccacgatggtactgatgtcggtatctgggttaacggcccgttcgcgcacctgttcaccggcgtttacgaggagaactatatcccgcacgctctggcttacgcggcatgtgttggcactggtcgtacgttctgcgacgaaaaataatgaaagcttgcggccgcactcgag

SEQ ID NO: 4 - protein, 969 aa, Artificial Sequence
  Chimeric Fusion Proteins
MMNDGKQQSTFLFHDYETFGTHPALDRPAQFAAIRTDSEFNVIGEPEVFYCKPADDYLPQPGAVLITGITPQEARAKGENEAAFAARIHSLFTVPKTCILGYNNVRFDDEVTRNIFYRNFYDPYAWSWQHDNSRWDLLDVMRACYALRPEGINWPENDDGLPSFRLEHLTKANGIEHSNAHDAMADVYATIAMAKLVKTRQPRLFDYLFTHRNKHKLMALIDVPQMKPLVHVSGMFGAWRGNTSWVAPLAWHPENRNAVIMVDLAGDISPLLELDSDTLRERLYTAKTDLGDNAAVPVKLVHINKCPVLAQANTLRPEDADRLGINRQHCLDNLKILRENPQVREKVVAIFAEAEPFTPSDNVDAQLYNGFFSDADRAAMKIVLETEPRNLPALDITFVDKRIEKLLFNYRARNFPGTLDYAEQQRWLEHRRQVFTPEFLQGYADELQMLVQQYADDKEKVALLKALWQYAEEIVRTGGSGGASGGSGGHMEEDKAYWNKDAQDALDKQLGIKLREKQAKNVIFFLGDGMSLSTVTAARIYKGGLTGKFEREKISWEEFDFAALSKTYNTDKQVTDSAASATAYLTGVKTNQGVIGLDANTVRTNCSYQLDESLFTYSIAHWFQEAGRSTGVVTSTRVTHATPAGTYAHVADRDWENDSDVVHDREDPEICDDIAEQLVFREPGKNFKVIMGGGRRGFFPEEALDIEDGIPGEREDGKHLITDWLDDKASQGATASYVWNRDDLLAVDIRNTDYLMGLFSYTHLDTVLTRDAEMDPTLPEMTKVAIEMLTKDENGFFLLVEGGRIDHMHHANQIRQSLAETLDMEEAVSMALSMTDPEETIILVTADHGHTLTITGYADRNTDILDFAGISDLDDRRYTILDYGSGPGYHITEDGKRYEPTEEDLKDINFRYASAAPKHSVTHDGTDVGIWVNGPFAHLFTGVYEENYIPHALAYAACVGTGRTFCDEK

(19) United States
(12) Patent Application Publication
(10) Pub. No.: US 2016/0230156 A1
(43) Pub. Date: Aug. 11, 2016

(54) RECOMBINANT FUSION PROTEIN POSSESSING NUCLEASE AND PHOSPHATASE ACTIVITY

(71) Applicant: LIFE TECHNOLOGIES CORPORATION, Carlsbad, CA (US)

(72) Inventor: Stephen HENDRICKS, Los Gatos, CA (US)

(21) Appl. No.: 14/776,856

(22) PCT Filed: Mar. 17, 2014
(86) PCT No.: PCT/US14/30515

§ 371 Date: Sep. 15, 2015

Related U.S. Application Data
(60) Provisional application No. 61/798,671, filed on Mar. 15, 2013.

(51) Int. Cl. C12N 9/22; C12N 9/16

(52) U.S. Cl. CPC C12N 9/22; C12N 9/16; C07K 2319/00; C12Y 301/11001; C12Y 301/03001

(57) ABSTRACT

Disclosed herein is a fusion protein possessing both nuclease and phosphatase activities. The described fusion protein simplifies the processing of amplified DNA to degrade residual primers and nucleotide triphosphates and thereby facilitates subsequent DNA analysis.

### CROSS REFERENCE TO RELATED APPLICATIONS

This application is a U.S. National Application filed under 35 U.S.C. 371 of PCT Application Serial No. PCT/US2014/030515 filed Mar. 17, 2014, which claims a priority benefit under 35 U.S.C. §119(e) from U.S. Provisional Application No. 61/798,671 filed Mar. 15, 2013, which is incorporated herein by reference.

### BACKGROUND

Nucleases are useful reagents for removing unwanted nucleic acids from protein preparations. Descriptions of certain nucleases and their preparation by cloning are provided by Eaves et al. (J. Bacteriol. 85:273-8 (1963)), Filimonova et al. (Biochem. Mol. Biol. Int. 33(6):1229-36 (1994)), Ball et. al. (Gene 57(2-3):183-92 (1987), Molin et. al. (U.S. Pat. No. 5,173,4185), and Friedhoff et al. (Protein Expr. Purif. 5(1):37-43 (1994)).

Exonuclease I (Exo I) digests single-stranded DNA in a 3′ to 5′direction producing 5′ mononucleotides. This enzyme is particularly useful in preparing amplified DNA products, such as PCR products, for sequencing. It degrades residual primers from the amplification reaction that would otherwise be carried over into the sequencing reaction. U.S. Pat. Nos. 5,741,676 and 5,756,285 generally disclose methods for DNA sequencing via amplification, both of which are hereby incorporated herein by reference. (See also R. L. Olsen et al., Comp. Biochem. Physiol., vol. 99B, No. 4, pp. 755-761 (1991)).

Amplification primers carried over into a sequencing reaction could act as sequencing primers and generate sequencing reaction products, thereby creating a background of secondary sequences which would obscure or interfere with observing the desired sequence. Both the concentration and specific activity (purity) of commercially available Exonuclease I may vary over a wide range. Commonly the enzyme is manufactured to a specific activity between 50,000 and 150,000 units of enzyme per mg and supplied for the purpose of processing amplified DNA at a concentration around 10 units per microliter. Enzyme with either higher or lower specific activity and either more or less concentrated could be employed in the described applications by suitable alterations in the applied protocol, such as adding less or more volume (or amount) of enzyme, respectively.

Alkaline Phosphatases, as exemplified by Shrimp Alkaline Phosphatase (SAP) and Calf Intestinal Alkaline Phosphatase (CIP), catalyze the hydrolysis of 5′-phosphate residues from DNA, RNA, and ribo- and deoxyribonucleoside triphosphates (dNTPs or nucleotide triphosphates). SAP is particularly useful in preparing amplified products, such as PCR products, for sequencing because it can readily be inactivated by heat prior to performing a sequencing reaction. SAP degrades residual dNTPs from the amplification reaction. If residual dNTPs are carried over from the amplification reaction to the sequencing reaction, they add to, and thereby alter, the concentration of dNTPs in the sequencing reaction in an indeterminant and non-reproducible fashion. Since, within narrow limits, high quality sequencing requires specific ratios between the sequencing reaction dNTPs and ddNTPs, an alteration in the concentration of dNTPs may result in faint sequencing reaction signals.

Prior to sequencing or other analyses, Exo I and SAP are frequently used to process PCR reaction products. There is a need in the art for an improved composition that provides both nuclease and phosphatase activity.

### SUMMARY OF THE INVENTION

Disclosed herein is a novel recombinant fusion protein. This fusion protein possesses two enzymatic activities, a nuclease activity and a phosphatase activity. Methods for the isolation and use of this novel protein are also disclosed.

### BRIEF DESCRIPTION OF THE DRAWINGS

FIG. 1 depicts a scheme for isolating the chimeric nuclease/phosphatase fusion protein.

FIG. 2 depicts a SDS-PAGE analysis at the indicated stages and fractions.

FIG. 3 depicts FIG. 3A a SDS-PAGE analysis of the indicated fraction, FIG. 3B a buffer volume and the absorbance at 260 nm and 280 nm at various buffer volumes witnessed on the POROS® HS cation exchange column, and FIG. 3C a buffer volume and the absorbance at 260 nm and 280 nm at various buffer volumes witnessed on the POROS® HQ cation exchange column.

FIG. 4 depicts FIG. 4A a SDS-PAGE analysis of fractions collected from the POROS® HS cation exchange column and indicates the fractions that were pooled, FIG. 4B a buffer volume and the absorbance at 260 nm and 280 nm at various buffer volumes witnessed on the POROS® HS cation exchange column, FIG. 4C a SDS-PAGE analysis of fractions collected from the POROS® HQ cation exchange column and indicates the fractions that were pooled, and FIG. 4D a buffer volume and the absorbance at 260 nm and 280 nm at various buffer volumes witnessed on the POROS® HQ cation exchange column.

FIG. 5 depicts the Quality Value (QV) score, which represents a per-base estimate of base call accuracy, and electropherograms at the indicated molar concentration of chimeric exonuclease/phosphatase fusion protein and controls. (A) ExoSAP-IT® QV (B) ExoSAP-IT® electropherogram (C) chimeric exonuclease/phosphatase fusion protein 8 μM QV (D) chimeric exonuclease/phosphatase fusion protein 8 μM electropherogram (E) chimeric exonuclease/phosphatase fusion protein 4 μM QV (F) chimeric exonuclease/phosphatase fusion protein 4 μM electropherogram (G) chimeric exonuclease/phosphatase fusion protein 2 μM QV (H) chimeric exonuclease/phosphatase fusion protein 2 μM electropherogram (I) no enzyme control QV (J) no enzyme control electropherogram.

FIG. 6 depicts an electropherogram from a MicroSeq® reaction using ExoSAP-IT®.

FIG. 7 depicts an electropherogram for a MicroSeq® reaction using the recombinant fusion protein (SEQ ID NO. 2) in place of ExoSAP-IT®.

FIG. 8 depicts an electropherogram from a MicroSeq® “fast” reaction using ExoSAP-IT®.

FIG. 9 depicts an electropherogram from a MicroSeq® “fast” reaction using the recombinant fusion protein (SEQ ID NO. 2) in place of ExoSAP-IT®.

### DETAILED DESCRIPTION OF THE INVENTION

In some embodiments are provided a recombinant fusion protein. “Recombinant” or “recombinant nucleic acid” or “recombinant gene” or “recombinant DNA molecule” or “recombinant nucleic acid sequence” indicates that the nucleotide sequence or arrangement of its parts is not a native configuration, and has been manipulated by molecular biological techniques. The term implies that the DNA molecule is comprised of segments of DNA that have been artificially joined together, for example, the polynucleotide encoding a nuclease and a phosphatase activity disclosed herein. Protocols and reagents to manipulate nucleic acids are common and routine in the art (See e.g., Maniatis et al. (eds.), Molecular Cloning: A Laboratory Manual, Cold Spring Harbor Laboratory Press, NY, 1982; Sambrook et al. (eds.), Molecular Cloning: A Laboratory Manual, Second Edition, Volumes 1-3, Cold Spring Harbor Laboratory Press, NY, 1989; and Ausubel et al. (eds.), Current Protocols in Molecular Biology, Vol. 1-4, John Wiley & Sons, Inc., New York 1994; all of which are herein incorporated by reference).

“Fusion protein” refers to a polypeptide composed of a plurality of components, unjoined in their native state but are joined to form a single continuous polypeptide.

Similarly, a “recombinant protein” or “recombinant polypeptide” refers to a protein molecule that is expressed from a recombinant DNA molecule. Use of these terms indicates that the primary amino acid sequence, arrangement of its domains or nucleic acid elements which control its expression are not native, and have been manipulated by molecular biology techniques. As indicated above, techniques to manipulate recombinant proteins are also common and routine in the art.

As used herein, the word “protein” refers to a full-length protein, a portion of a protein, or a peptide. Proteins can be produced via fragmentation of larger proteins, or chemically synthesized. Proteins may, for example, be prepared by recombinant overexpression in a species such as, but not limited to, bacteria, yeast, insect cells, and mammalian cells. Proteins to be placed in a protein microarray of the invention, may be, for example, are fusion proteins, for example with at least one affinity tag to aid in purification and/or immobilization. In certain aspects of the invention, at least 2 tags are present on the protein, one of which can be used to aid in purification and the other can be used to aid in immobilization. In certain illustrative aspects, the tag is a His tag, a GST tag, or a biotin tag. Where the tag is a biotin tag, the tag can be associated with a protein in vitro or in vivo using commercially available reagents (Invitrogen, Carlsbad, Calif.). In aspects where the tag is associated with the protein in vitro, a Bioease tag can be used (Invitrogen, Carlsbad, Calif.).

As used herein, the term “peptide,” “oligopeptide,” and “polypeptide” are used interchangeably with protein herein and refer to a sequence of contiguous amino acids linked by peptide bonds. As used herein, the term “protein” refers to a polypeptide that can also include post-translational modifications that include the modification of amino acids of the protein and may include the addition of chemical groups or biomolecules that are not amino acid-based. The terms apply to amino acid polymers in which one or more amino acid residue is an analog or mimetic of a corresponding naturally occurring amino acid, as well as to naturally occurring amino acid polymers. Polypeptides can be modified, for example, by the addition of carbohydrate residues to form glycoproteins. The terms “polypeptide,” “peptide” and “protein” include glycoproteins, as well as non-glycoproteins.

In some embodiments are provided a recombinant fusion protein, wherein the recombinant fusion protein possesses two enzymatic activities, wherein the first enzymatic activity is a nuclease activity.

“Nuclease” refers to an enzyme capable of cleaving phosphodiester bonds between the nucleotide subunits of nucleic acids.

In some embodiments are provided a recombinant fusion protein, wherein the recombinant fusion protein possesses two enzymatic activities, wherein the first enzymatic activity is a nuclease activity and the nuclease activity is exonuclease activity.

“Exonuclease” refers to an enzyme that cleaves nucleotides one at a time from the end of a polynucleotide chain via a hydrolyzing reaction that breaks phosphodiester bonds at either the 3′ or 5′ end. The “exonuclease” can be a 3′ to 5′ exonuclease or a 5′ to 3′ exonuclease. E. coli exonuclease I and exonuclease III are two commonly used 3′-exonucleases that have 3′-exonucleolytic single-strand degradation activity. E. coli exonuclease VII and T7-exonuclease Gene 6 are two commonly used 5′-3′ exonucleases that have 5′-exonucleolytic single-strand degradation activity.

Exonucleases can be originated from prokaryotes, such as E. coli exonucleases, or eukaryotes, such as yeast, worm, murine, or human exonucleases. Examples of exonucleases that can be used in the disclosed fusion protein include, but are not limited to, E. coli exonuclease I, E. coli exonuclease III, E. coli exonuclease VII, bacteriophage lambda exonuclease, and bacteriophage T7-exonuclease Gene 6, or a combination thereof.

In some embodiments are provided a recombinant fusion protein, wherein the recombinant fusion protein possesses two enzymatic activities, wherein the first enzymatic activity is a nuclease activity and the second enzymatic activity is a phosphatase activity.

“Phosphatase” or “alkaline phosphatase” refers to an enzyme capable of hydrolyzing phosphoric monoesters to produce inorganic phosphoric acids. Alkaline phosphatases are generally known to be metal-dependent enzymes that have low substrate specificity and require metal ions such as magnesium ions (Mg²⁺) or zinc ions (Zn²⁺) for enzymatic reactions. Typical alkaline phosphatases include bacterial alkaline phosphatase (BAP), calf intestinal alkaline phosphatase (CIAP), shrimp alkaline phosphatase (SAP) and the like.

In some embodiments are provided a nucleic acid encoding a recombinant fusion protein, wherein the recombinant fusion protein possesses two enzymatic activities, wherein the first enzymatic activity is a nuclease activity and the second enzymatic activity is a phosphatase activity.

“Nucleic acid” refers to polymers of single or double stranded nucleotide. A nucleic acid typically refers to a polynucleotide molecule comprised of a linear strand of two or more nucleotides (deoxyribonucleotides and/or ribonucleotides) or variants, derivatives and/or analogs thereof

In some embodiments the nucleic acid encompasses SEQ ID NO:1.

SEQ ID NO. 1:
ACAAATAGCGTCCAAGAGAACATTGT
AATGAATCCTTCTGATAATTGTCTGATGAGCGATGTCATGAAAGTCAACC
TGTCTCGTCGTAAAATTCTGCAGTTTGGCGGGGCAATGGGTGCTATCGCC
CTGCTGCCTGCGGCTTTTGGTTCGCGCAGCGCATTTGCGAACCCAAGCCA
GCCATTGCAGATCCGTAAAATTACAAATCTCACGTTTACGTCTATTCCGT
TTAGTACCGAAGATCGTGTCATCGTTCCAAAAGGCTATAGTGCGAAGGCG
TTCTATGCCTGGGGCGACCCGGTCGGTCTGGAAAATAATAACCCGCAGTT
TAAAGTTGACGCGTCAAACTCGGCAGAAGAACAGGCCGCTCAGGCGGGCA
TGAACCACGATGGGATGGCGTACTTTCCTTTCGCCGAACATGGTAACGAA
CATGGCCTGCTGGTGATGAACCATGAATACATTGACAACGGTCTCTTGTT
TCCTGATGGGGATAAAACGTGGAGCCTGGATAAGGTGAAAAAATCGCAGA
ACGCTATGGGCATCTCGGTGATTGAAATTAAAAAAGTGAACCAGCAGTGG
GAAGTGGTCCGCCCGAGCAAATATGCCCGTCGTATTACGCCGCATACCCC
TATGCGTCTGACAGGGCCGGCGAAACATAACGAACTGATGAAAACTGTAG
CCGACCCTTTAGGGGAGTTTGTGCTGGGGACCATGCAGAACTGTGCAAAT
GGTGAAACACCCTGGCGCACTTATCTGACCTGTGAAGAGAACTGGTCCGA
TATTTTTGTGCGTGAATCCGGCGACTTCACGAAGCTGGATAAGCGTTACG
GGATTATGAAGAAAGAAAAAGAAGATAAGTACCGTTGGAATGAATTCGAT
GAACGTTTTAATACGGATAAACATCCGAACGAACCGCACCGCTTCGGCTG
GGTTGTTGAAATTGACCCCTTTGATCCAAACAGCACCCCGGTTAAACACA
CCGCCCTGGGCCGCTTCAAACACGAGGGCGCCATGCTGGTGCTGAGCAAG
GAAGGGCACGCAGTAGTCTACATGGGTGACGATCAGCGTTTCGAGTACAT
CTATAAGTTCGTTTCTAAAGGTAAATACAACCCGGCAGATCGTGCAGCAA
ACATGTATCTTCTGTCCGAAGGCACACTCTATGTCGCCCATTTTAACGAA
GATAACACCGGCGAATGGCGTCCGCTGGTCCATAATCAGAACGGGCTGAC
TGCGGAGAATGGCTTTCTGAATCAGGGCGATGTGACCATCAAGGCACGTA
TGGCGGCTGATGTGGTGGGCGGCACTAAAATGGATCGCCCGGAATGGATC
GCCGTCGATCCATATCAGACTGGCTCTGTGTATTGCACCCTTACAAACAA
TAGCCAGCGTGGTACCGAAGGCAAGGCGGGTATTGATGCCGCCAACCCGC
GCGTTAAGAACTCGTATGGACATATCATTCGCTGGCAAGAGAATGACCAA
GATTACCTGTCCGAGACCTTCTCATGGGATATCTTCGCGCTGGGCGGTAA
TAAATCCAAGGGCGAAAATCATGTGAACGGTGATGATTTCGGTTCCCCGG
ACGGACTCCGTTTCGATAATCACGGCATCTTGTGGGTTCAAACGGACGTG
TCGAGTTCCACGTTAAATAAAAAGGCCTATGAAGGCATGGGTAATAACCA
AATGCTGGCGGTAATTCCAGAGCAGGGCGAGTTTAAACGTTTTTTAACGG
CCCCGAACGGCTCAGAAGTAACCGGTATCGCTTTCACTCCTGACAACAAA
ACCATGTTTATCAATATTCAGCATCCGGGTGAACCAGATAGCGGCGTGAC
CGAACCAGATCAAGTAACAGCCATTTCCACCTGGCCGGACCGTCAAGGTA
AAACCCGCCCTCGCTCTGCTACCGTGGTTATTCAGAAGGAAGATGGTGGC
GTTATTTCATCTCTCGAGCACCACCACCACCACCAC

In some embodiments the recombinant fusion protein possessing both nuclease and phosphatase activity encompasses the polypeptide sequence of SEQ ID NO:2.

SEQ ID NO: 2
MMNDGKQQSTFLFHDYETFGTHPALDRPAQFAAIRTDSEFNVIGEPEVFY
CKPADDYLPQPGAVLITGITPQEARAKGENEAAFAARIHSLFTVPKTCIL
GYNNVRFDDEVTRNIFYRNFYDPYAWSWQHDNSRWDLLDVMRACYALRPE
GINWPENDDGLPSFRLEHLTKANGIEHSNAHDAMADVYATIAMAKLVKTR
QPRLFDYLFTHRNKHKLMALIDVPQMKPLVHVSGMFGAWRGNTSWVAPLA
WHPENRNAVIMVDLAGDISPLLELDSDTLRERLYTAKTDLGDNAAVPVKL
VHINKCPVLAQANTLRPEDADRLGINRQHCLDNLKILRENPQVREKVVAI
FAEAEPFTPSDNVDAQLYNGFFSDADRAAMKIVLETEPRNLPALDITFVD
KRIEKLLFNYRARNFPGTLDYAEQQRWLEHRRQVFTPEFLQGYADELQML
VQQYADDKEKVALLKALWQYAEEIVRTGGSGGGSGGGSGTNSVQENIVMN
PSDNCLMSDVMKVNLSRRKILQFGGAMGAIALLPAAFGSRSAFANPSQPL
QIRKITNLTFTSIPFSTEDRVIVPKGYSAKAFYAWGDPVGLENNNPQFKV
DASNSAEEQAAQAGMNHDGMAYFPFAEHGNEHGLLVMNHEYIDNGLLFPD
GDKTWSLDKVKKSQNAMGISVIEIKKVNQQWEVVRPSKYARRITPHTPMR
LTGPAKHNELMKTVADPLGEFVLGTMQNCANGETPWRTYLTCEENWSDIF
VRESGDFTKLDKRYGIMKKEKEDKYRWNEFDERFNTDKHPNEPHRFGWVV
EIDPFDPNSTPVKHTALGRFKHEGAMLVLSKEGHAVVYMGDDQRFEYIYK
FVSKGKYNPADRAANMYLLSEGTLYVAHFNEDNTGEWRPLVHNQNGLTAE
NGFLNQGDVTIKARMAADVVGGTKMDRPEWIAVDPYQTGSVYCTLTNNSQ
RGTEGKAGIDAANPRVKNSYGHIIRWQENDQDYLSETFSWDIFALGGNKS
KGENHVNGDDFGSPDGLRFDNHGILWVQTDVSSSTLNKKAYEGMGNNQML
AVIPEQGEFKRFLTAPNGSEVTGIAFTPDNKTMFINIQHPGEPDSGVTEP
DQVTAISTWPDRQGKTRPRSATVVIQKEDGGVISSLEHHHHHH

In some embodiments are provided a vector with a nucleic acid insert, wherein the nucleic acid insert encodes a recombinant fusion protein, wherein the recombinant fusion protein possesses two enzymatic activities, wherein the first enzymatic activity is a nuclease activity and the second enzymatic activity is a phosphatase activity. In other embodiments, the nucleic acid insert encompasses SEQ ID NO:1. In some embodiments, the nucleic acid encodes the polypeptide of SEQ ID NO:2.

“Vector” refers to any DNA or RNA molecule that acts as an intermediate carrier into which a DNA or RNA segment is inserted for introduction into a host cell for amplification. Such intermediate carriers include plasmids, cosmids, bacteriophages and transposons.

“Host cell” refers to any cell type which is susceptible to transformation, transfection, and/or transduction with a nucleic acid construct. A host cell can be a prokaryotic or eukaryotic cell.

In some embodiments, the recombinant fusion protein is purified.

The terms “purified” and “isolated” as used herein, are synonymous, and refer to a material that is substantially or essentially free from other components. For example, in one embodiment, a recombinant protein is isolated or purified when it is free from other components used in the cloning reaction, or solid state synthesis, isolation or purity is generally determined using analytical chemistry techniques such as polyacrylamide gel electrophoresis, mass spectrometry, or high performance liquid chromatography (HPLC). In one embodiment, a polynucleotide, protein or peptide of the present invention is considered to be isolated when it is the predominant species present in a preparation. A purified protein, peptide or nucleic acid molecule of the invention represents greater than about 80% of the macromolecular species present, greater than about 90% of the macromolecular species present, greater than about 95% of the macromolecular species present, greater than about 96% of the macromolecular species present, greater than about 97% of the macromolecular species present, greater than about 98% of the macromolecular species present, greater than about 99% of the macromolecular species present in a preparation.

In some embodiments are provided a kit encompassing a recombinant fusion protein, wherein the recombinant fusion protein possesses two enzymatic activities, wherein the first enzymatic activity is a nuclease activity and the second enzymatic activity is a phosphatase activity.

The kits of the present invention may also comprise instructions for performing one or more methods described herein and/or a description of one or more compositions or reagents described herein. Instructions and/or descriptions may be in printed form and may be included in a kit insert. A kit also may include a written description of an internet location that provides such instructions or descriptions.

### EXAMPLES

### Construction of Fusion Protein

At the outset, several chimeric fusion proteins possessing both nuclease and phosphatase activity were envisioned and tested. For instance, the combination of exonuclease and shrimp alkaline phosphatase activity of SEQ ID NO. 3 and SEQ ID NO. 4 was constructed and tested. This chimeric fusion protein failed in early stages of development for lacking solubility and desired enzymatic activity. A number of other chimeric exonuclease/phosphatase fusion proteins were also tested but failed.

SEQ ID NO 3:
AGATCTCGATCCCGCGAAATTAATACGACTCACTATAGGGGAATTGTGAGCGGATA
ACAATTCCCCTCTAGAAATAATTTTGTTTAACTTTAAGAAGGAGATATACATATGAT
GAATGATGGTAAACAGCAATCTACCTTTCTGTTCCACGACTATGAAACCTTCGGTAC
TCACCCGGCCCTGGATCGCCCGGCACAGTTCGCTGCGATCCGTACCGACTCCGAATT
TAACGTGATCGGTGAACCGGAGGTATTCTACTGCAAACCGGCGGACGACTACCTGC
CACAGCCGGGTGCGGTGCTGATTACCGGTATCACTCCGCAAGAAGCTCGTGCCAAA
GGTGAGAATGAAGCGGCGTTTGCCGCGCGTATCCATAGCCTGTTCACCGTACCGAAA
ACCTGCATCCTGGGTTACAACAACGTGCGTTTCGACGACGAAGTGACCCGTAACATC
TTCTACCGTAACTTCTATGACCCATACGCATGGTCCTGGCAGCACGACAACAGCCGT
TGGGATCTGCTGGATGTAATGCGTGCGTGCTATGCTCTGCGCCCAGAAGGTATTAAC
TGGCCGGAGAACGACGACGGCCTGCCGAGCTTCCGTCTGGAGCACCTGACCAAAGC
GAACGGTATCGAACACTCCAACGCGCACGATGCGATGGCAGACGTCTATGCTACTA
TCGCTATGGCAAAGCTGGTTAAAACCCGTCAGCCGCGCCTGTTTGACTATCTGTTTA
CCCACCGTAACAAACACAAACTGATGGCTCTGATCGACGTTCCGCAGATGAAGCCG
CTGGTTCATGTGTCTGGTATGTTTGGTGCTTGGCGCGGCAACACCTCTTGGGTAGCCC
CGCTGGCCTGGCACCCGGAGAACCGTAACGCTGTGATCATGGTGGACCTGGCGGGT
GATATCTCCCCGCTGCTGGAACTGGACTCTGACACGCTGCGTGAACGTCTGTATACC
GCAAAAACCGATCTGGGTGATAACGCCGCAGTTCCGGTGAAGCTGGTGCACATCAA
CAAATGTCCGGTCCTGGCTCAGGCGAATACCCTGCGTCCGGAAGACGCGGACCGTC
TGGGTATTAACCGTCAGCATTGCCTGGACAACCTGAAAATTCTGCGCGAAAACCCGC
AGGTCCGCGAAAAAGTTGTAGCCATCTTCGCGGAAGCGGAACCGTTTACCCCATCC
GACAACGTTGACGCTCAGCTGTACAACGGCTTCTTTTCCGATGCGGACCGCGCAGCG
ATGAAAATTGTTCTGGAAACCGAACCGCGCAACCTGCCGGCACTGGATATCACTTTC
GTCGACAAACGTATCGAAAAACTGCTGTTCAACTATCGTGCTCGTAACTTTCCGGGT
ACTCTGGATTACGCTGAGCAACAGCGTTGGCTGGAACATCGTCGTCAGGTATTTACC
CCGGAATTCCTGCAGGGCTATGCAGATGAACTGCAGATGCTGGTACAACAGTACGC
AGACGATAAGGAGAAAGTGGCGCTGCTGAAAGCACTGTGGCAGTACGCGGAAGAA
ATTGTTCGTACGGGCGGCTCCGGTGGCGCGAGCGGCGGTTCCGGCGGTCATATGGA
AGAAGATAAAGCATACTGGAACAAAGACGCGCAGGATGCCCTGGACAAACAGCTG
GGTATCAAACTGCGTGAAAAACAGGCCAAAAACGTGATTTTCTTCCTGGGTGATGGT
ATGAGCCTGTCCACGGTTACTGCGGCGCGTATCTATAAAGGCGGTCTGACTGGTAAA
TTCGAACGTGAAAAAATCTCTTGGGAAGAGTTCGACTTCGCAGCCCTGTCTAAAACT
TATAATACGGATAAACAGGTTACGGATTCTGCTGCTTCTGCAACCGCTTATCTGACC
GGCGTTAAGACCAACCAGGGTGTTATTGGTCTGGACGCTAACACCGTTCGTACCAAC
TGCTCTTACCAGCTGGATGAAAGCCTGTTTACCTACAGCATCGCACACTGGTTCCAG
GAAGCTGGTCGCAGCACCGGTGTTGTGACCTCCACCCGTGTTACCCACGCTACTCCG
GCGGGCACCTACGCGCACGTAGCAGATCGCGATTGGGAAAACGACAGCGACGTAGT
ACATGATCGTGAAGACCCGGAAATTTGTGACGATATCGCAGAACAGCTGGTATTCC
GTGAGCCGGGCAAAAACTTTAAAGTAATCATGGGTGGCGGTCGTCGCGGTTTCTTCC
CGGAAGAAGCGCTGGACATCGAAGATGGTATCCCGGGTGAGCGTGAAGACGGTAAA
CACCTGATCACTGACTGGCTGGATGACAAGGCTTCCCAGGGTGCAACTGCATCCTAC
GTATGGAACCGTGATGACCTGCTGGCGGTGGACATCCGCAACACTGATTACCTGATG
GGCCTGTTCAGCTACACGCACCTGGACACCGTTCTGACCCGTGATGCCGAAATGGAC
CCGACTCTGCCTGAGATGACTAAAGTGGCCATCGAAATGCTGACCAAAGACGAAAA
TGGTTTCTTTCTGCTGGTAGAAGGCGGTCGCATTGACCACATGCACCACGCGAACCA
GATCCGTCAGTCTCTGGCTGAGACCCTGGACATGGAGGAGGCCGTTAGCATGGCGCT
GAGCATGACTGATCCGGAAGAAACGATCATCCTGGTTACCGCTGATCACGGTCATAC
GCTGACTATCACCGGTTACGCGGACCGTAACACGGATATTCTGGATTTCGCTGGCAT
CAGCGATCTGGACGACCGTCGCTACACTATCCTGGATTACGGTTCTGGTCCGGGTTA
CCACATCACTGAGGACGGCAAACGCTACGAACCGACTGAAGAGGATCTGAAAGATA
TCAATTTCCGCTACGCGTCTGCAGCACCAAAACATTCTGTTACCCACGATGGTACTG
ATGTCGGTATCTGGGTTAACGGCCCGTTCGCGCACCTGTTCACCGGCGTTTACGAGG
AGAACTATATCCCGCACGCTCTGGCTTACGCGGCATGTGTTGGCACTGGTCGTACGT
TCTGCGACGAAAAATAATGAAAGCTTGCGGCCGCACTCGAG
SEQ ID NO. 4
MMNDGKQQSTFLFHDYETFGTHPALDRPAQFAAIRTDSEFNVIGEPEVFYCKPADDYLP
QPGAVLITGITPQEARAKGENEAAFAARIHSLFTVPKTCILGYNNVRFDDEVTRNIFYRNF
YDPYAWSWQHDNSRWDLLDVMRACYALRPEGINWPENDDGLPSFRLEHLTKANGIEH
SNAHDAMADVYATIAMAKLVKTRQPRLFDYLFTHRNKHKLMALIDVPQMKPLVHVSG
MFGAWRGNTSWVAPLAWHPENRNAVIMVDLAGDISPLLELDSDTLRERLYTAKTDLG
DNAAVPVKLVHINKCPVLAQANTLRPEDADRLGINRQHCLDNLKILRENPQVREKVVAI
FAEAEPFTPSDNVDAQLYNGFFSDADRAAMKIVLETEPRNLPALDITFVDKRIEKLLFNY
RARNFPGTLDYAEQQRWLEHRRQVFTPEFLQGYADELQMLVQQYADDKEKVALLKAL
WQYAEEIVRTGGSGGASGGSGGHMEEDKAYWNKDAQDALDKQLGIKLREKQAKNVIF
FLGDGMSLSTVTAARIYKGGLTGKFEREKISWEEFDFAALSKTYNTDKQVTDSAASATA
YLTGVKTNQGVIGLDANTVRTNCSYQLDESLFTYSIAHWFQEAGRSTGVVTSTRVTHAT
PAGTYAHVADRDWENDSDVVHDREDPEICDDIAEQLVFREPGKNFKVIMGGGRRGFFP
EEALDIEDGIPGEREDGKHLITDWLDDKASQGATASYVWNRDDLLAVDIRNTDYLMGL
FSYTHLDTVLTRDAEMDPTLPEMTKVAIEMLTKDENGFFLLVEGGRIDHMHHANQIRQS
LAETLDMEEAVSMALSMTDPEETIILVTADHGHTLTITGYADRNTDILDFAGISDLDDRR
YTILDYGSGPGYHITEDGKRYEPTEEDLKDINFRYASAAPKHSVTHDGTDVGIWVNGPF
AHLFTGVYEENYIPHALAYAACVGTGRTFCDEK

Purification of Chimeric Enzyme from Bacterial Host Cells

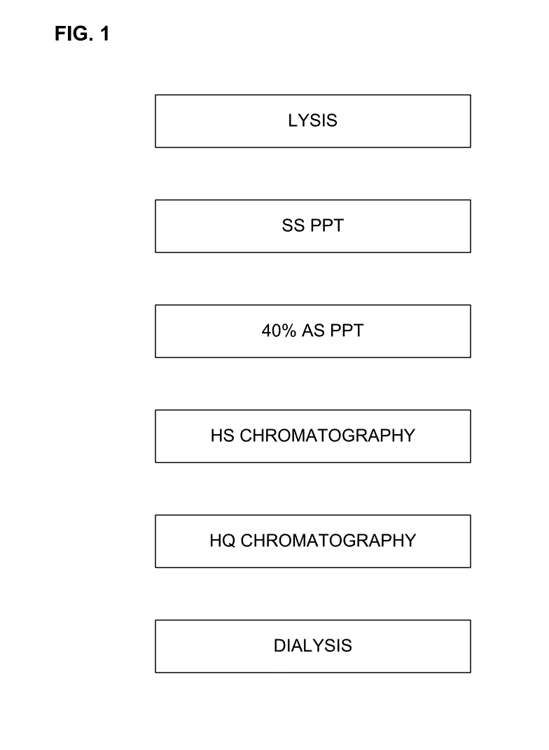

In order to isolate the fusion protein (SEQ ID NO. 2) the following procedure was used, schematically represented in FIG. 1. Escherichia coli cells harboring an expression plasmid with the chimeric exonuclease/phosphatase fusion protein as an insert were cultured at 37° C. to an optical density (OD) of 0.5 when measured at 600 nm (OD₆₀₀). At this point the culture temperature was reduced to 18° C. and 1 mM of isopropyl β-D-1-thiogalactopyranoside (IPTG) was added to induce expression of the chimeric exonuclease/phosphatase fusion protein. Expression was allowed to proceed overnight at 18° C. Following this, the cells were subjected to centrifugation and then lysed using a high pressure Microfluidizer®. The resultant lysate was treated with 2% streptomycin sulfate and centrifuged at 19,000 rpm for 30 minutes at 4° C.

To precipitate the chimeric exonuclease/phosphatase fusion protein, after centrifugation 40% saturated ammonium sulfate was added to the supernatant and then this was subjected to centrifugation. The resulting pellet is resuspended in 25 mM HEPES (pH 6.3).

The resuspended pellet was then loaded onto a POROS® HS cation exchange column equilibrated with 25 mM HEPES (pH 6.3). Peak fractions from a 0-1 M NaCl gradient were pooled and diluted 1:2 prior to loading onto a POROS® HQ anion exchange column equilibrated with 25 mM HEPES (pH 6.3). Peak fractions were pooled and dialyzed against 25 mM HEPES (pH 8.2), 2 mM CaCl₂ and 50% glycerol.

This methodology resulted in approximately 3 mg. of the expressed chimeric exonuclease/phosphatase fusion protein per gram of bacteria.

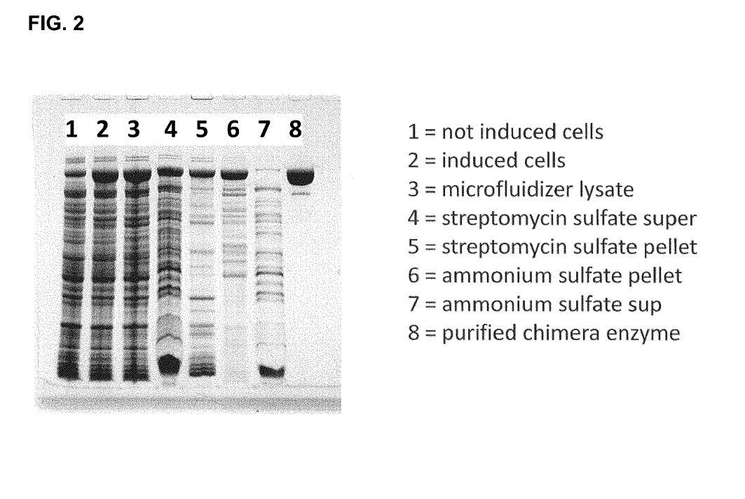

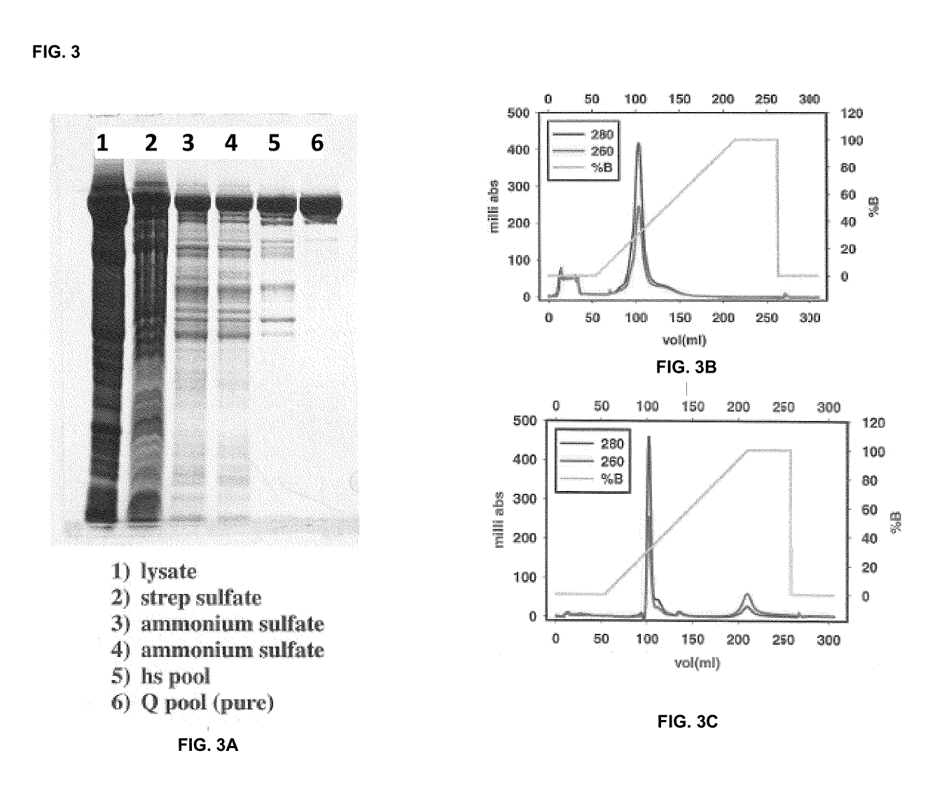

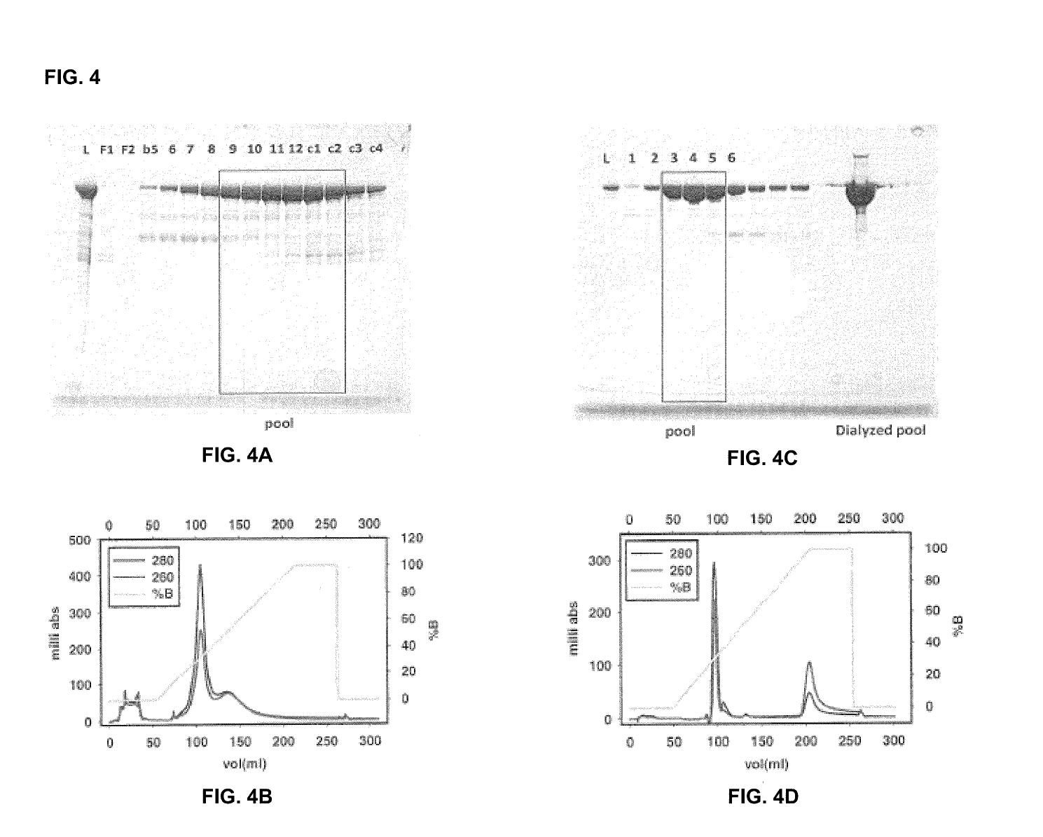

At a number of steps in the purification process, samples were taken and analyzed by SDS-PAGE. Results of these analyses are depicted in FIGS. 2, 3 and 4.

### Analysis of the Chimeric Exonuclease/Phosphatase Fusion Protein in Functional Assays

The chimeric exonuclease/phosphatase fusion protein (SEQ ID NO. 2) was utilized in a DNA sequencing workflow to assess its functional characteristics. Human genomic DNA was used as template for PCR amplification of amplification of 639 bp portion of the HLA locus. After the completion of thermal cycling, various molar concentrations of the chimeric exonuclease/phosphatase fusion protein were added to the PCR amplification tubes and the reactions were incubated for 15 minutes at 37° C., followed by 15 minutes at 80° C. ExoSAP-IT® was added to some amplification tubes as a comparative control.

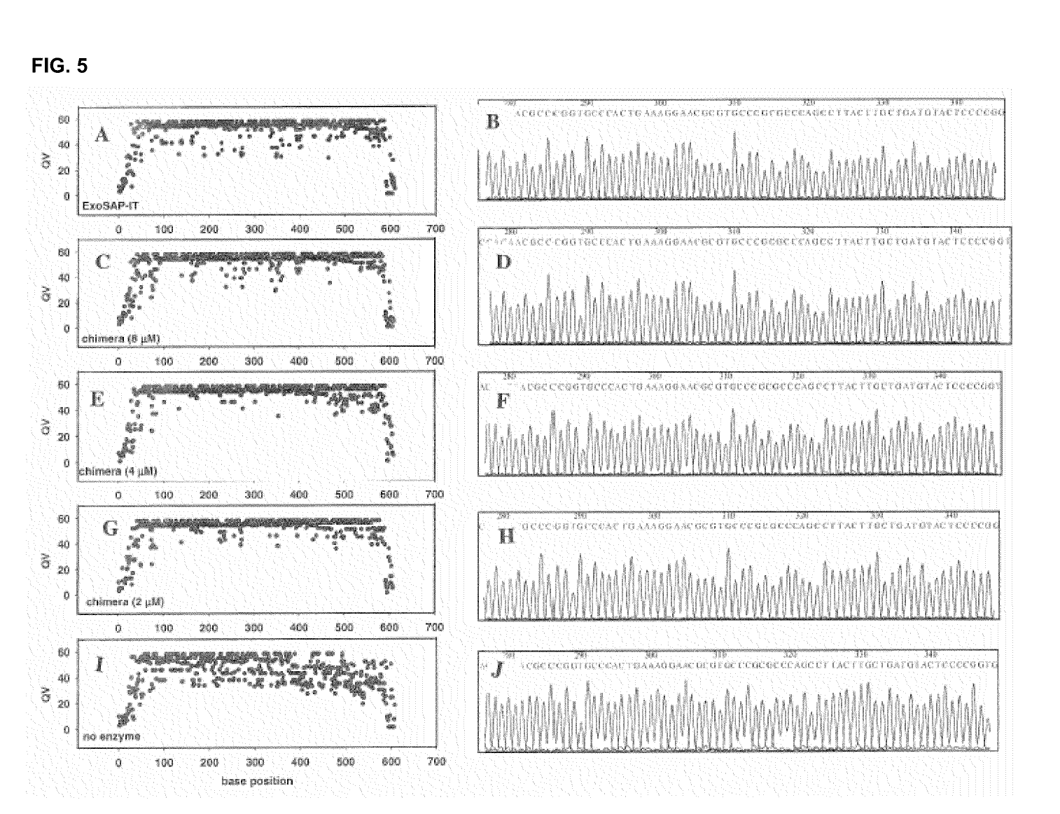

After the incubation was complete, each reaction was subjected to DNA cycle sequencing. Resulting QV and electropherograms from the cycling sequencing are depicted in FIG. 5.

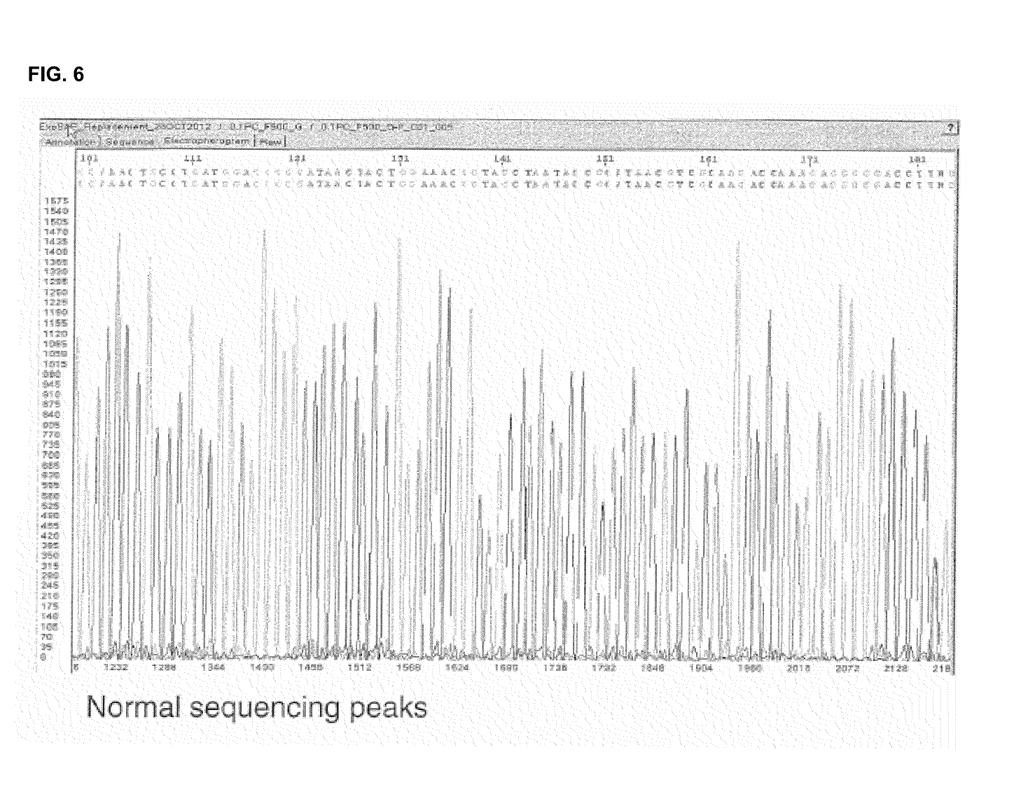

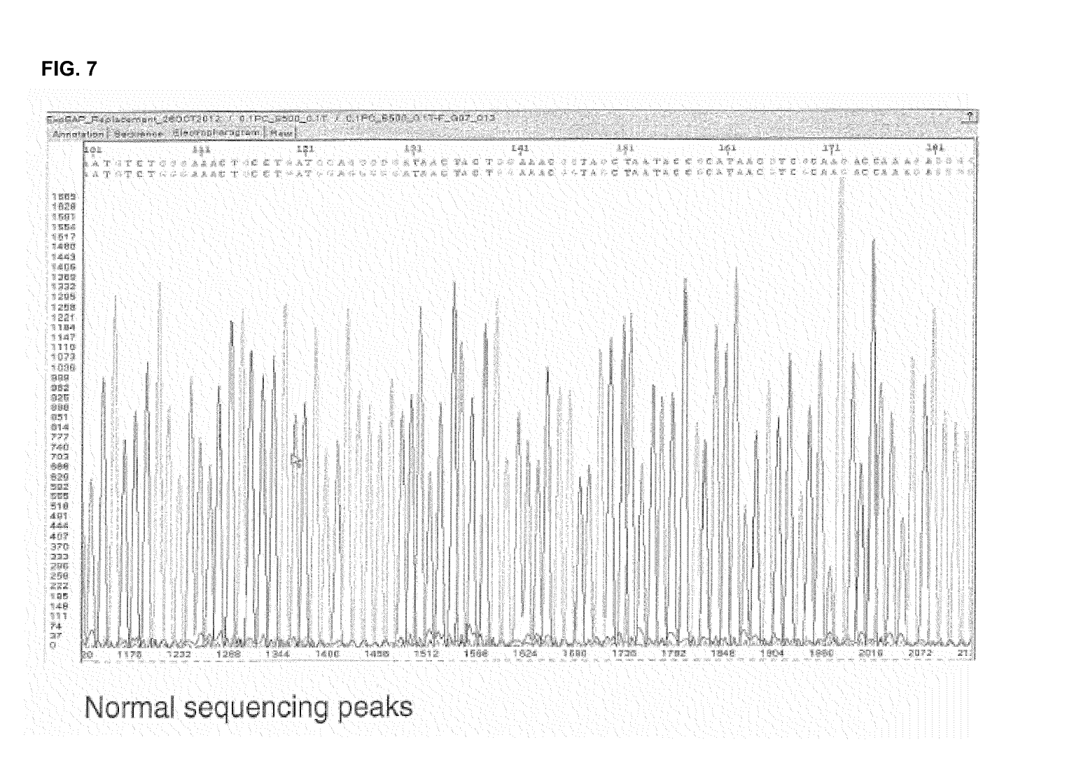

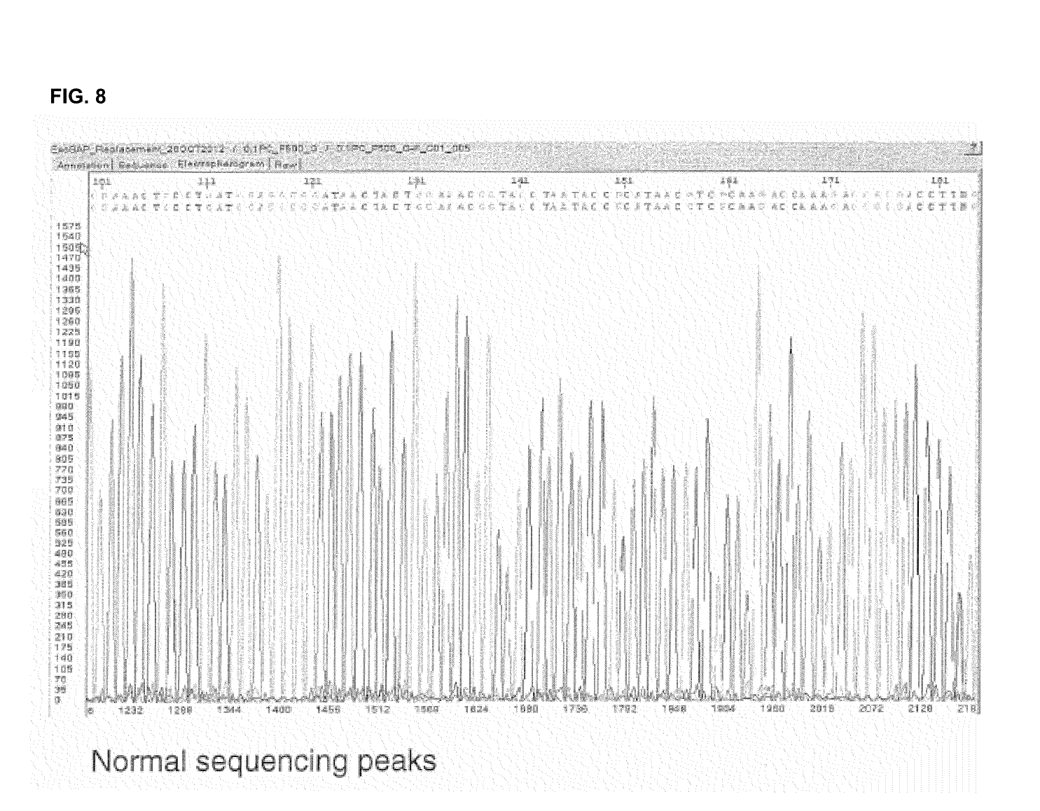

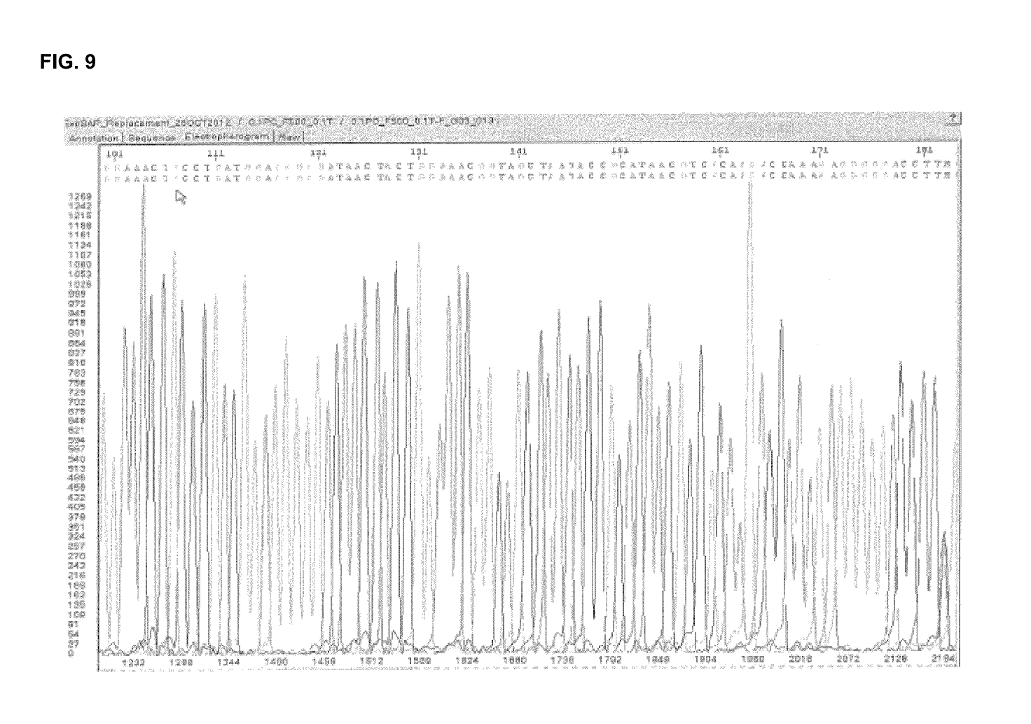

In addition to the human HLA locus DNA sequencing was performed using a bacterial 16S ribosomal RNA gene (rDNA) sequence as a template. To accomplish this, an approximately 500 bp amplicon was generated by PCR amplification of a bacterial rDNA sequence. After PCR amplification, the chimeric exonuclease/phosphatase fusion protein was added to the reaction and incubated, followed by cycle sequencing and gel electrophoresis. Results from representative experiments using the protein of SEQ ID NO. 2 are depicted in FIGS. 6-9.

we claim:

1. A composition comprising a recombinant fusion protein, wherein the recombinant fusion protein possesses two enzymatic activities, wherein the first enzymatic activity is a nuclease activity and the second enzymatic activity is a phosphatase activity.

2. The composition of claim 1, wherein the nuclease activity is an exonuclease activity.

3. A nucleic acid encoding the recombinant fusion protein of claim 1.

4. A vector comprising the nucleic acid of claim 3.

5. A host cell comprising the nucleic acid of claim 3.

6. The nucleic acid of claim 1, wherein the nucleic acid sequence comprises SEQ ID NO:1.

7. A method comprising expressing a recombinant fusion protein within a host cell, wherein the recombinant fusion protein possesses two enzymatic activities, wherein the first enzymatic activity is a nuclease activity and the second enzymatic activity is a phosphatase activity and obtaining the recombinant fusion protein from the host cell.

8. A kit comprising a single container comprising a recombinant fusion protein, wherein the recombinant fusion protein possesses two enzymatic activities, wherein the first enzymatic activity is a nuclease activity and the second enzymatic activity is a phosphatase activity.

9. The composition of claim 1, wherein the recombinant fusion protein is substantially purified.
